(19) United States
(12) Patent Application Publication
(10) Pub. No.: US 2014/0194747 A1
(43) Pub. Date: Jul. 10, 2014

(54) INFRARED SCANNER AND PROJECTOR TO INDICATE CANCEROUS CELLS

(71) Applicants: Lewis John Kruglick, Escondido, CA (US); Ezekiel Kruglick, Poway, CA (US)

(72) Inventors: Lewis John Kruglick, Escondido, CA (US); Ezekiel Kruglick, Poway, CA (US)

(73) Assignee: EMPIRE TECHNOLOGY DEVELOPMENT LLC, Wilmington, DE (US)

(21) Appl. No.: 13/884,116

(22) PCT Filed: May 1, 2012
(86) PCT No.: PCT/US12/36008

§ 371 Date: May 8, 2013

(51) Int. Cl. A61B 5/00; A61B 1/06; A61B 1/313; A61B 1/07

(52) U.S. Cl. CPC A61B 5/0086; A61B 1/07; A61B 1/0638; A61B 1/313
USPC 600/473; 600/178

(57) ABSTRACT

Provided herein are methods and devices for detecting and/or indicating cancerous cells. In some embodiments, infrared light can be used to induce an infrared signature of one or more cells and visible light can be used to indicate the one or more cells having the infrared signature.

### TECHNICAL FIELD

Some embodiments herein generally relate to apparatus and methods for detecting and indicating cancerous cells.

### BACKGROUND

A variety of methods exist for selecting cancerous cells for surgical removal. Existing surgical intervention typically involves taking obvious tumors plus a safety margin which can result in the loss of a substantial amount of the tissue.

### SUMMARY

In some embodiments, an endoscopic probe, laparoscopic probe, or endoscopic and laparoscopic probe is provided. The probe can include at least one light guide including an input and an output. The at least one light guide allows infrared and visible light to pass through the light guide. The light guide further includes a mirror assembly in optical communication with the light guide. The mirror assembly is configured to (a) direct an infrared beam from the light guide, (b) receive an infrared signature and direct it into the light guide, and (c) direct a visible light beam from the light guide.

In some embodiments, a system for guiding and collecting light is provided. The system can include a probe. The probe can include a light guide and an optical head connected to the light guide. The optical head can optionally be detachable. The system further includes a collinear light guide that is configured to be in optical communication with the probe and an infrared (IR) light source. The infrared light source is configured to be in optical, communication with the collinear light guide. The infrared light source can optionally be configured to be in detachable communication with the collinear light guide. The system further includes a visible light source that is configured to be in optical, communication with the collinear light guide, and a detector. The visible light source can optionally be configured to be in detachable communication with the collinear light guide. The system is configured to allow the detector to detect infrared light that enters the system through the light guide.

In some embodiments, a method for indicating a target cell is provided. The method can include detecting an infrared signature from one or more cells and projecting at least one wavelength of visible light onto an area corresponding to the one or more cells, thereby indicating a target cell.

### BRIEF DESCRIPTION OF THE DRAWINGS

FIG. 1 is a flowchart depicting some embodiments of a method of indicating a target cell.

FIGS. 2A-C are spectrographic plots depicting some embodiments of infrared signatures.

FIG. 3 is a flowchart depicting some embodiments of a method of indicating a target cell.

FIG. 4 is a drawing depicting some embodiments of a system for guiding and collecting light.

FIG. 5 is a photograph depicting an example of some embodiments of indicating target cells.

FIG. 6 is a flow chart depicting some embodiments of how the method can be performed.

FIG. 7 is a drawing depicting some embodiments of a computing system.

FIG. 8 is a drawing depicting some embodiments of a program product.

FIG. 9 is a drawing depicting some embodiments of a computing system.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Provided herein are embodiments that generally relate to the detection and indication (and/or visualization) of particular cell types (e.g., cancerous cells). A combination of cell state detection (e.g., is a cell cancerous) and image projection (e.g., illuminating a section of tissue that contains the cancerous cell to allow visualization of the cancerous area) is provided. The two are configured to be, or can be, provided during a medical process, such as the manipulation and/or removal of a cancerous tissue. Some embodiments provided herein can be implemented in and/or as a laparoscopic or endoscopic probe. Detection can be performed by spectroscopy and the coupling between detection and indication of cancerous cells can involve collinear beams for spectroscopy and identification before one or both beams passes through an optical head. The spectroscopy and image beams can be bounced off the same optical head (for example, a scanning mirror assembly), which can provide for further advantages. In some embodiments, the tissue to be examined can be liver tissue and the examination can allow for a superior identification of the resection margin.

Indicating a target cell can include detecting an infrared signature from one or more cells and projecting at least one wavelength of visible light onto an area corresponding to the one or more cells to thereby identify (or indicate) the target cell or cells.

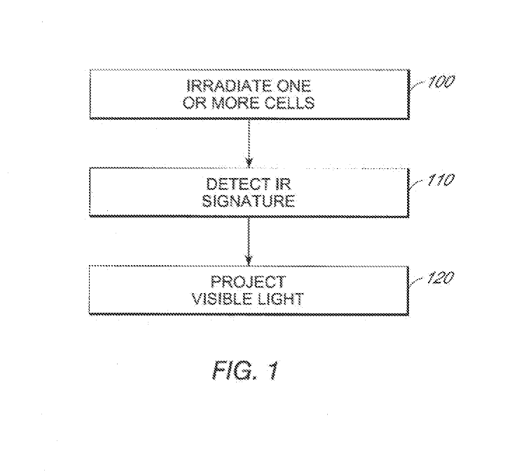

FIG. 1 is a flow chart that depicts some embodiments of a method of indicating a target cell. The method can include irradiating one or more cells (block 100) and detecting an infrared signature from the one or more cells (block 110). The infrared signature can indicate which, if any, of the irradiated cells has an IR signature that is cancerous and/or of interest. The method can further include projecting at least one wavelength of visible light (block 120) onto the one or more cells so as to selectively indicate which areas contain cancerous cells (or other cells of interest) and which areas do not. Thus, one both detects cancerous cells (via their IR signature or other optical mechanism) and indicates the area(s) that those cells are located in on a subject (or tissue) by of the use of visible light. This allows for a practitioner to observe any remaining cancerous tissue or cells, during manipulation of the cells. This can be employed, for example, during the removal of a cancerous section of tissue, allowing for a greater degree of certainty that all of the cancerous tissue has been removed. The various wavelengths of light (IR and/or visible) can pass through a same optical head and/or optical probe, allowing for one or more of tissue irradiation, IR signature detection, and/or tissue indication to be done by a relatively small probe.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

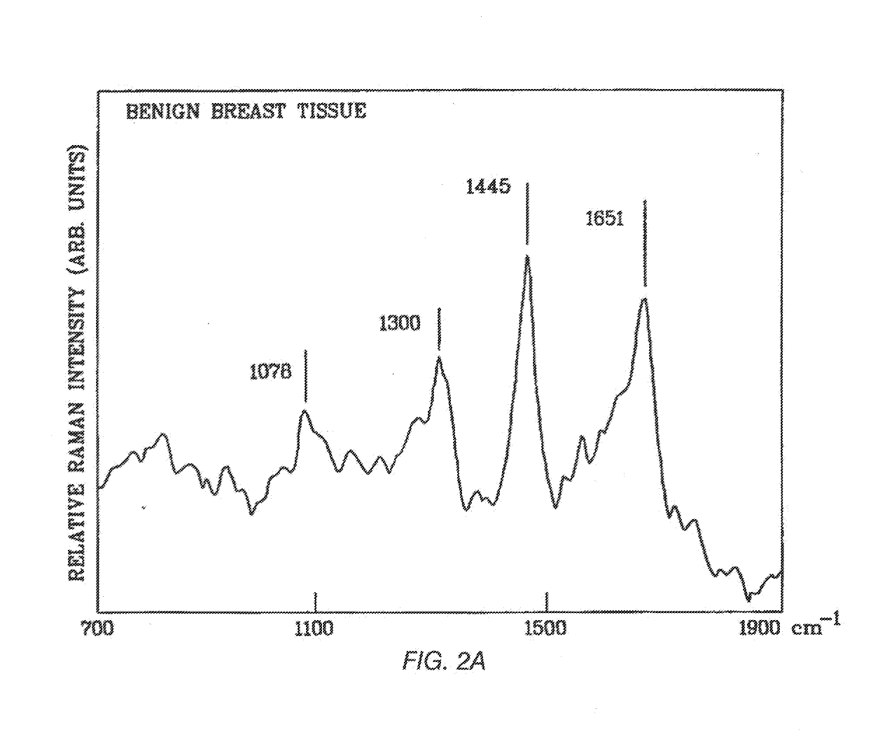

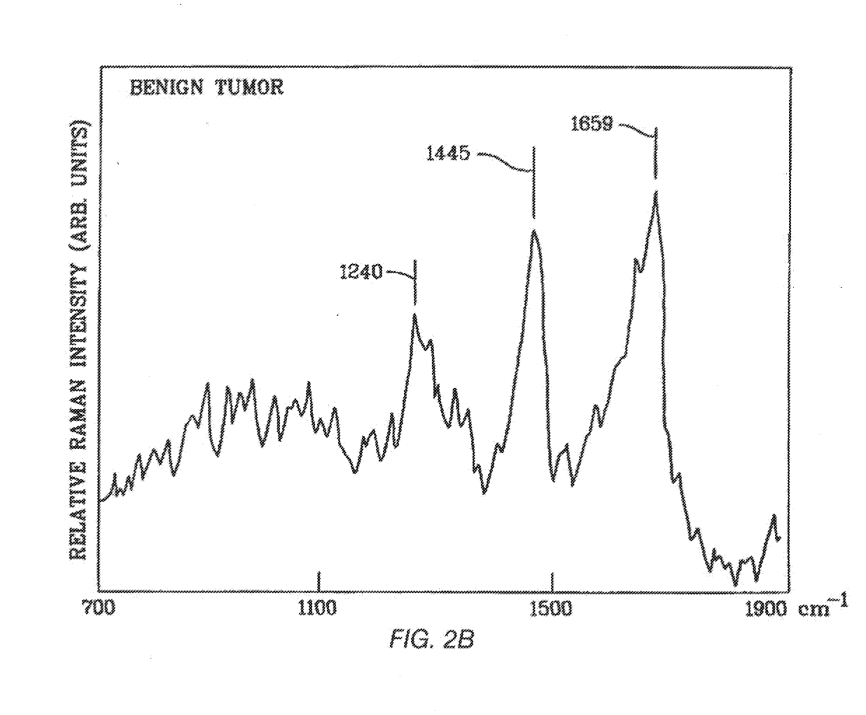

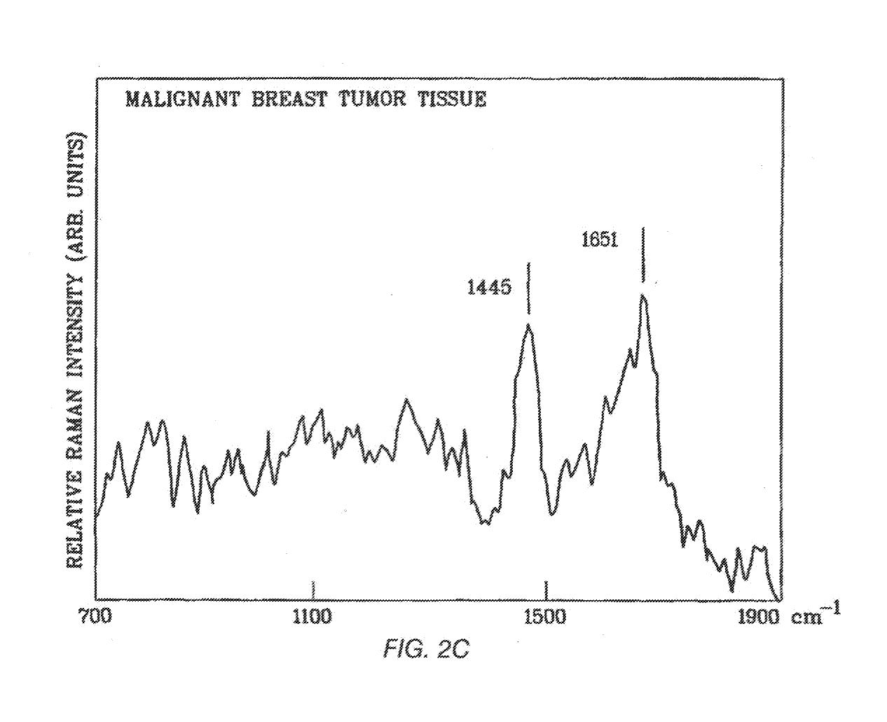

Detecting the infrared signature includes detecting an optical characteristic emitted by one or more cells. The optical characteristic is any optical characteristic that allows one to detect some aspect of the cells and/or distinguish a first cell population from a second cell population. The optical characteristic can be the emission properties of cells that have been irradiated with infrared light. The optical characteristic (or “signature”) of the cells when irradiated by an infrared light is used in any number of ways. It can be used to identify (and/or distinguish between) cells that are pre-cancerous, benign, and/or malignant. As the internal biochemical differences between malignant and non-malignant cells show up when irradiated with infrared light, it has been established that IR spectroscopy can be used to analyze tissues to determine whether or not a particular section is normal, pre-cancerous, benign tumor or malignant tumor. Exemplary IR signatures from various tissue types are shown in FIGS. 2A-2C. One can also identify what type, size, depth, etc., of a cancer section of cells. This can be achieved by observing the IR signature produced, the change in IR signature produced (in comparison to a control sample) and/or the comparison of the IR signature (and/or its change) to one or more IR signatures of various known and/or control samples. A variety of techniques and methods exist for the detection of cancerous and other cellular states for various cells. The present embodiments are not limited to any particular approach or technique, and any IR (or other radiation) based detection system can be employed in some of the present embodiments.

As shown in FIG. 1, visible light can be used to indicate the specific location of a particular cell type (or cellular state) by projecting the light onto the target cell (block 120). Projecting the visible light can be done by a detachably connected optical head. In some embodiments, the projected light indicates the target cell. In some embodiments, the projected light indicates the non-targeted cell (so that the target cells are indicated as not being illuminated within an illuminated area). In some embodiments, white light is projected and used as the indicator of the target cell. In some embodiments, one or more wavelengths of visible light can be selectively projected onto the target cell, thereby indicating the target cell and/or providing additional information regarding the target cell and/or its surroundings.

In some embodiments, visible light is simply used to indicate an area of a target cell. The visible light can be provided as an image and/or include more than simply an illuminated area. The wavelength of the visible light can be selected so as to be different from other wavelengths of light around the area of interest (for example, other visible light that might be projected by the method, ambient light on the tissue, and/or surgical light). The wavelength of light of the at least one wavelength of visible light can be selected so as to be different than any wavelength of light projected on the one or more cells that are not the target cell. The wavelength of light of the at least one wavelength of visible light can be selected so as to be visibly distinguishable from any wavelength of light projected on the one or more cells that are not the target cell. In some embodiments, a wavelength of light of the at least one wavelength of visible light is selected so as to contrast with an environment around the one of more cells. In some embodiments, the wavelength of light for illumination includes and/or emphasizes blue, yellow, or blue and yellow wavelength(s). In some embodiments, the wavelength of visible light corresponds to information to be provided to a practitioner. In some embodiments, the wavelength can correspond to a size of a cancer cluster. For example, in some embodiments, the wavelength of visible light can correspond to the average diameter of the cancer cluster. In some embodiments, the wavelength of the visible light corresponds to a depth of a cancerous cell. The depth of the cancerous cell or cells can correspond to a flight time of the IR signature of the cell. The visible light can be provided as a particular shape (e.g. an arrow, a square, a star, a ring, a circle, etc.) In some embodiments, the visible light is provided as a structured image. In some embodiments, the visible light can be projected so as to include text. In some embodiments, the visible light can simply be projected as a representation of the location of the target cells. Thus, in some embodiments, the visible light can effectively provide an image of target cells or target cell clusters and/or a tumorous mass. A map of the IR signatures from an area of tissue being examined can be turned into a corresponding visible light map (or image) and this image can be projected onto the tissue or cells. In some embodiments, the image or visible light map can also be registered by the image system for tracking so that the image stays in place if the tool or body moves.

As will be appreciated by one of skill in the art, the illumination of a “target cell” does not denote that the illumination itself needs be specific and/or exclusive to the cellular level, but merely that the illumination occur for at least the target cell. Thus, illuminating a target cell can encompass illuminating non-target cells proximal to the target cell as well. As will be appreciated by one of skill in the art, the illuminated area, indicating the target cell, can be focused such that excessive areas of healthy tissue are not indicated by the illumination, so that excessive levels of healthy tissue are not needlessly removed. However, as it is frequently more important to remove all of the cancerous tissue, some general illumination of the surrounding healthy cells can occur in some embodiments, so as to make certain that all of the target cells are removed. In some embodiments, the amount of the neighboring healthy tissue that can be illuminated is 2 cm or less from the cancerous and/or undesired cell and/or tissue, for example, an illuminated zone that is less than 2, 1.5, 1, 0.5, 0.3, 0.2, or 0.1 cm wide can surround the target cell and/or target area.

In some embodiments, the visible light image that is projected includes two or more wavelengths of visible light. In some embodiments, projecting at least one wavelength of visible light includes projecting at least a first wavelength of visible light onto a first cell and at least a second wavelength of visible light onto a second cell. The first wavelength of light can be different from the at least second wavelength of light. In some embodiments, the first cell is a cancerous cell. In some embodiments, the first cell is a part of a tumor tissue. In some embodiments, the second cell is a non-cancerous cell. In some embodiments, the second cell is a part of a benign tissue. In some embodiments, the second cell is a non-cancerous cell and the second wavelength of light is different from the first wavelength of light. As noted above, the resolution need not be at the cellular level, and can instead be at the tissue level (and the designation of a “first cell” and/or “a second cell” includes designating clusters of cells and/or areas of tissue that include the cells), as long as at least one cell in the “cancerous tissue” is cancerous and at least one cell in the healthy tissue (or other tissue) is healthy. In some embodiments, any of the cell based descriptions provided herein can be applied to a tissue level application, where clusters of cells are indicated and/or areas of tissue are indicated. The disclosure provided herein should not be taken as indicating that single cell resolution is required for any of the herein provided embodiments.

In some embodiments, different types of cancerous cells can be identified by different wavelengths of visible light. In some embodiments, different sizes of cancer clusters can be identified by different wavelengths of visible light. In some embodiments different depths of cancer clusters can be identified by different wavelengths of visible light.

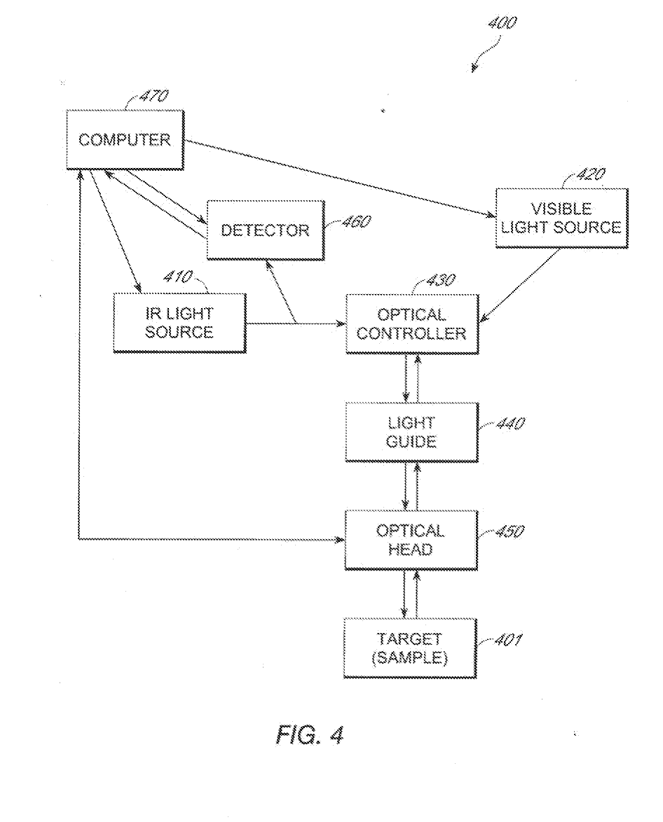

The visible light can be generated by a visible light source (such as an arc lamp, a halogen bulb, a diode, a laser, etc.), and the visible light passes into a collinear light guide, into a probe light guide, and then to the optical head (see the schematic of FIG. 4).

In some embodiments, the wavelength of visible light is from about 380 nm to about 750 nm, for example, the visible light has a wavelength of 380, 400, 420, 440, 460, 480, 500, 520, 540, 560, 580, 600, 620, 640, 660, 680, 700, 720, 740, or 750 nm, including any range between any two of the preceding values. In some embodiments, the visible light is blue, yellow, or blue and yellow. In some embodiments, the light source can be a diode. In some embodiments, the at least one wavelength of visible light is configured to be white light and/or colored light. In some embodiments, more than one wavelength of light is employed, e.g., 0.1, 1, 2, 5, 10, 15, 20, 30, 40, 50, 60, 70, 80, 90, 95, 98, 99, or 100% of the visible light spectrum can be used, including any range between any two of the preceding values and any range beneath any one of the preceding values.

As shown in FIG. 1, in some embodiments, the method includes irradiating one or more cells (block 100) with at least one wavelength of infrared light. The method can include irradiating the one or more cells with at least one wavelength of infrared light to thereby induce the one or more cells to provide the infrared signature, which can then be detected and used to locate which areas contain target cells and/or which areas do not contain target cells.

The infrared light can be generated by an infrared light source, and the at least one wavelength of infrared light passes into a collinear light guide, into a probe light guide, to the optical head, and to (and then from) the tissue. In some embodiments, the light guide and/or optical head can be used to both transmit IR light from the light source to the tissue, as well as gather light (e.g., the IR signature from the tissue) and direct it for processing of the IR information to determine which areas of the tissue have IR signatures that are of interest.

The wavelength of infrared light is from about 0.7 μm to about 80 μm. In some embodiments, the at least one wavelength of infrared light has a wavelength of 0.7, 1, 10, 50, 100, 200, 300, 400, 500, 600, 700, 800, 900, 950, 990, 1000 μm, including any range between any two of the preceding values. In some embodiments, more than one wavelength of IR light is employed, e.g., 0.1, 1, 2, 5, 10, 15, 20, 30, 40, 50, 60, 70, 80, 90, 95, 98, 99, or 100% of the IR light spectrum can be used, including any range between any two of the preceding values and any range beneath any one of the preceding values.

In some embodiments, the infrared light used for irradiating the one or more cells and the visible light, both pass through the same light guide. The infrared light and the visible light can be collimated prior to entering the optical head. This can be achieved by employing a prism, a dichroic reflector, or other optical device. In some embodiments, the light and the visible light are collimated before entering the probe light guide. In some embodiments, the infrared light and the visible light are collimated in the collinear light guide.

In some embodiments, the collinear light guide, probe light guide, and optical head that the infrared light passes through is the same collinear light guide, probe light guide, and optical head that the visible light passes through. In some embodiments, the infrared signature passes through the same light guide as the infrared light and/or the visible light. In some embodiments, not only does the infrared signature pass through these components, but the location of the cancerous (or other target cells) is preserved as it passes through the parts of the system, thus, allowing one to create a map of the relative position of the target cells by the various optical properties from the various cells. One can then use the IR signature map to create a corresponding visible light map, which can then be projected onto the tissue and/or cells.

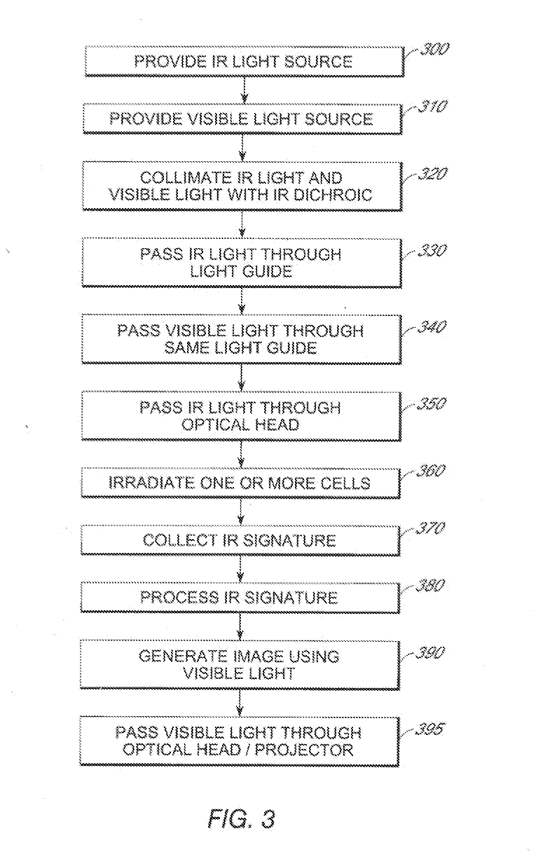

A method for indicating a target cell is provided (FIG. 3). The method can include providing an IR light source (block 300), that generates an IR light beam, providing a visible light source (block 310) that generates a visible light beam, and collimating the IR light beam and the visible light beam (block 320). The method can further include passing the IR light beam through a light guide (block 330), passing the visible light beam through the same light guide as the IR light beam (block 340), and passing the IR light beam through an optical head (block 350). The method can further include irradiating one or more cells with the IR light beam (block 360), the cells provide an IR signature as a result of the IR light beam, and collecting the IR signature (block 370) (which, in some embodiments, can be done via the optical head, which can direct the IR signature to an IR light detection system). The method can further include processing the IR signature (block 380), generating an image using the visible light beam (which can corresponds to the IR signature so as to allow the indication of target cells by the visible light beam) (block 390), and passing the visible light beam (projected image) through the optical head and/or projector (block 395). The visible light image can then be projected onto the cells from which the IR information came from, such that target cells (e.g., cancerous cells) are selectively indicated.

In some embodiments, an endoscopic probe, laparoscopic probe, or endoscopic and laparoscopic probe is provided. The probe can include at least one light guide including an input and an output. The at least one light guide allows infrared and visible light to pass through the light guide. The light guide further includes a mirror assembly in optical communication with the light guide. The mirror assembly is configured to (a) direct an infrared beam from the light guide, (b) receive an infrared signature and direct it into the light guide, and (c) direct a visible light beam from the light guide.

The at least one light guide includes a first end and second end. The first end is opposite the second end. The second end of the light guide is configured to receive an input from a light source, such as an infrared light source. The second end of the light guide can be configured to receive an input from a visible light source. In some embodiments, the light guide is configured to receive an input from the infrared light source and the visible light source.

The first end of the light guide can be configured to be attached to and in optical communication with a mirror assembly. The first end of the light guide can be configured to direct the light source input to the mirror assembly. The first end of the light guide can be configured to receive an output from the mirror assembly. The first end of the light guide can be configured to receive an IR signature.

In some embodiments, the mirror assembly includes at least one microelectromechanical system (MEMS) scanning mirror assembly. The optical head of the probe can be any device or component that allows one to selectively direct IR and/or visible light. A single light directing device (e.g., scanning mirror) can be configured to direct both the IR (both to irradiate and as emitted from the cells) and the visible light.

In some embodiments, a single light guide is used to guide the IR light (IR beam and/or IR signature) and the visible light. In some embodiments, the probe includes a single light guide. In some embodiments, the probe includes a second light guide. In some embodiments, light guide includes a collinear light guide (where the IR light from the IR light source and the visible light are collinear) and/or a probe light guide (which can be positioned before the optical head).

In some embodiments, the light guide includes a first light guide section, a second light guide section, and a third light guide section. The first light guide section can be configured to direct the infrared light beam. The second light guide section can be configured to direct the visible light beam. The first light guide section and the second light guide section can be configured to collimate the infrared beam and the visible light beam into the third light guide section. The third light guide section can be configured to direct the collimated infrared and visible light beams to the optical head (e.g., mirror assembly).

The probe can include an optical controller. The optical controller can be configured to selectively allow a desired range of wavelengths of light to pass through the optical controller and reflect other wavelengths of light. The optical controller can include a dichroic filter, mirror and/or reflector. The optical controller can be configured to prevent IR light from the visible light source from entering the probe. In some embodiments, the optical controller is located elsewhere in the system.

A system for guiding light is provided. The system can include a probe and a collinear light guide, configured to be in optical communication with the probe. The system further includes an infrared (IR) light source that is configured to be in optical, communication with the collinear light guide (which can optionally be detachable). The system further includes a visible light source that is configured to be in optical, communication with the collinear light guide (which can optionally be detachable). The system further includes a detector. The system is configured to allow the detector to detect infrared light that enters the system through the light guide, and is configured to allow for a probe to irradiate a tissue or cell sample, collect IR radiation from the tissue or cell sample, and direct visible light back to a selected section of the tissue or cells.

As depicted in the schematic diagram of FIG. 4, the system 400 includes a light guide 440 and an optical head 450 that, optionally, can be detachably connected to the light guide 440. One or more of these can be included in a probe (which can be handheld). The system can also include an infrared (IR) light source 410. The infrared light source 410 can be configured to be in optical communication with the collinear light guide 440 (which can optionally be detachable). The system 400 can include a visible light source 420 that is configured to be in optical, communication with the collinear light guide 440 (which can optionally be detachable). The system 400 can include a detector 460. The system 400 can be configured to allow the detector 460 to detect infrared light that enters the system 400 through the light guide 440 (e.g., allows for the detection of the IR signature).

The infrared light source 410 can be any source and/or device capable of producing infrared light. In some embodiments, the infrared light source 410 is a light emitting diode and/or a laser diode. In some embodiments, the infrared light source is an IR spectrometry light source. In some embodiments, the IR light source does not produce visible light. In some embodiments, the IR light source does produce visible light, but a filter is used to reduce and/or remove the visible light, so it does not interfere with the projected visible light used to indicate the presence of the target cell(s).

The infrared light source 410 can be configured to produce infrared light having at least one wavelength from about 0.7 μm to about 1000 μm. The infrared light source 410 can be configured to produce infrared light having at least one wavelength of 0.7, 1, 10, 50, 100, 200, 300, 400, 500, 600, 700, 800, 900, 950, 990, or 1000 μm, including any range below any one of the preceding values, and any range between any two of the preceding values. In some embodiments, the infrared light source provides a range, scan, and/or sweep along a range of wavelengths over time, for example by adjusting a filtering of a broad wavelength source. Thus, various wavelengths (or spectrum with various peaks of infrared light) can be employed in some embodiments. In some embodiments, the device further includes a filter for these manipulations.

In some embodiments, the infrared light source 410 is pulsed. In some embodiments, the visible light source is pulsed. In some embodiments, a controller is set up such that when the IR light source is on, the visible light source is off. In some embodiments, this can be achieved by timing, without the need for a separate controller. In some embodiments, this allows for a single optical head to perform the process of directing the IR beam to the tissue, redirecting the IR signature from the tissue, into the rest of the system, and directing light from the system onto the target cells in a selective manner. In some embodiments, the IR source does not produce visible light, and/or the visible light is filtered out of the light.

The system 400 includes a visible light source 420. In some embodiments, the visible light source 420 is at least one light emitting diode or laser diode. In some embodiments, the visible light source 420 is configured to produce visible light having at least one wavelength from about 380 nm to about 750 nm. In some embodiments, the visible light source 420 is configured to produce visible light having at least one wavelength of 380, 400, 420, 440, 460, 480, 500, 520, 540, 560, 580, 600, 620, 640, 660, 680, 700, 720, 740, or 750 nm, including any range below any of the preceding values, any range above any of the preceding values, and any range between any two of the preceding values. In some embodiments, the visible light source does not produce IR light, and/or the IR light is filtered out of the light.

In some embodiments, the visible light source 420 is configured to produce white light. In some embodiments, the visible light source 420 is configured to produce colored light. In some embodiments, the device includes a prism for RGB collimation of the visible light.

The system 400 can include a probe. In some embodiments, the probe is as discussed herein. In some embodiments, the probe is a laparoscopic and/or endoscopic probe. In some embodiments, the probe is a surgical probe. In some embodiments, the probe includes a probe light guide and a collinear light guide.

The system 400 includes an optical head 450. The optical head 450 can be configured to direct infrared light from the probe light guide 440, receive an infrared signature from a cell and/or tissue and direct the infrared signature into the probe light guide 440, and/or direct visible light. The optical head 450 can be configured to receive an infrared signature from a target sample 401. In some embodiments, the mirror, and/or mirror array, can be flat. In some embodiments, the radius of curvature is greater than 50 cm. In some embodiments, the mirror and/or optical head can be any shape, for example, round, rectangular, hexagonal, octagonal, etc.

In some embodiments, the optical head 450 includes a scanning mirror assembly. In some embodiments, the optical head 450 includes at least one microelectromechanical system (MEMS) assembly. In some embodiments, the MEMS assembly directs sensing (e.g., IR) and indicating (e.g., visible) beams in coordination. In some embodiments, the optical head 450 is controlled and/or coordinated by a computer 470. In some embodiments, a single computer can control and/or coordinate the visible light source, IR Source, and/or optical head. The computer can also control and/or monitor the results from the detector 460. One or more computers and/or processors can be used to control one or more of these aspects. In some embodiments, the microelectromechanical system (MEMS) scanning mirror, and the system 400 are configured so that actuation of the MEMS scanning mirror is coordinated with pulsing of light from the visible light source 420. As noted herein, in some embodiments, the optical head (for example, via the scanning mirror) can be used for both scanning the IR and projecting the visible light. Thus, in some embodiments the visible light and IR light are coordinated. Coordination can allow for both the visible light and the IR to visit the same locations during a scanning cycle. In some embodiments, coordination is achieved by pulsing the visible light and/or IR light in a non-overlapping manner, with the actuation of the scanning mirror, so that both the visible light and the IR light can be appropriately projected and collected. This can allow one to use the optical head for providing the IR to the surface, collecting the IR signature, and projecting the visible light onto the surface. In some embodiments, as the optics can allow for overlapping (at the same time) use of both the IR light and the visible light, one can continuously scan the tissue (via IR light) while one displays the visible light created image.

In some embodiments, the optical head 450 includes a projector. In some embodiments, the projector is a picoprojector. In some embodiments, the projector is configured to project a visible image, including visible light, onto the area corresponding to one or more cells.

The system can include a detector 460. The detector 460 can detect infrared light that enters the system through the light guide 440. In some embodiments, the detector 460 detects the strength of the infrared signature, the frequency of the infrared signature, or both. In some embodiments, the detector detects the flight time of the infrared signature. In some embodiments, the flight time of the IR signature corresponds to the depth of the cell that provides the signature. In some embodiments, flight time can be measured by interfering the returning IR light with coherent light that has traveled a known distance along a reference path. Thus, in some embodiments, a reference path, having a known distance, in optical communication with at least a portion of the light path through which the returning IR light travels, is also provided.

In some embodiments, the detector 460 is a point detector. In some embodiments, the point detector includes a monochromator. In some embodiments, the point detector and monochromator can tune through frequencies and separate frequencies over time.

In some embodiments, the detector 460 includes a prism and/or grating. In some embodiments, the prism and/or grating splits the infrared signature into a local spectrum.

In some embodiments, the system 400 includes an image sensor. For example, in some embodiments, the detector 460 includes a charge coupled device (CCD). In some embodiments, the image sensor can include, but is not limited to, an active pixel sensor, a CCD, an intensified charge-coupled device (ICCD) or a complementary metal-oxide-semiconductor (CMOS).

The system 400 can include an optical controller 430. In some embodiments, the optical controller includes a filter and/or a mirror. In some embodiments, the filter is configured so that the detector 460 primarily receives infrared light. In some embodiments, filters can be employed so that visible light in the system does not interfere with the IR signature. The optical controller helps direct light from the IR light source and/or visible light from the visible light source. The filter can include at least one dichroic mirror configured to reflect infrared light while allowing visible light to pass through. The IR dichroic can make the IR spectrometer beam collinear with a visible image generating light. Any arrangement to make the IR beam collinear with the visible light beam can be employed.

The system can include a computing device 470. A computing device 470 can be used to process a spectroscopic signal and generate a desired and/or predicted visible image (e.g., a visible light map that correlates to the IR signatures obtained from the sample). The computing device 470 can be in communication with the detector 460. The computing device 470 can be in communication with the visible light source 420 and/or the IR light source 410. The computing device 470 can be in communication with a driver for the mirror assembly. The computing device 470 can control an amount of visible light that passes through the probe light guide 440.

The computing device 470 can be configured to control the optical head 450 such that a cell emitting an infrared signature consistent with a cancer is illuminated by visible light from the visible light source 420. The illumination can be achieved by the computing device 470 controlling the optical head 450 such that visible light from the visible light source 420 is directed to the cell, from which a cancerous IR signature previously (or currently) originated. For example this can be achieved by controlling the visible light from the visible light source 420 to a color indicating a cancerous state when a scanning mirror in the optical head 450 is at the same angle as it was previously in when the cancerous IR signature was detected. In this way the system does not need to know the absolute location of the cancerous IR signature, as indications can be given by reusing the same or similar optical path with visible light.

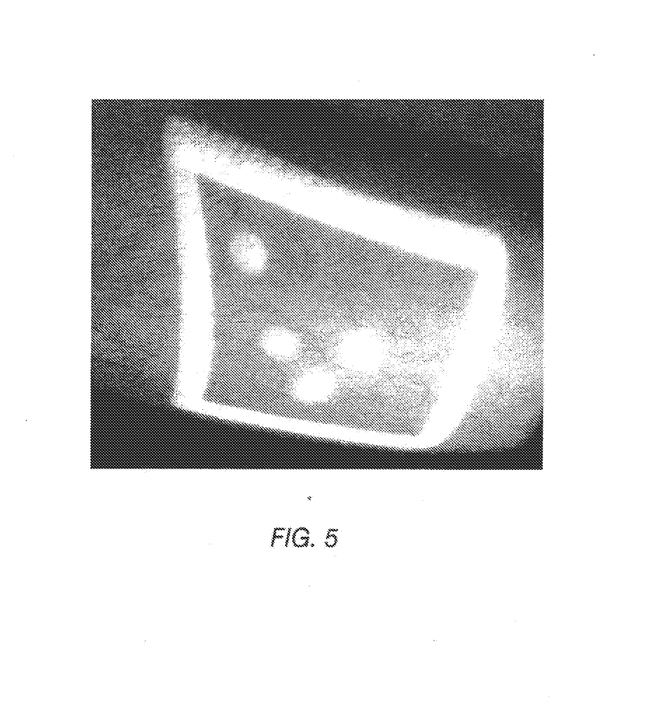

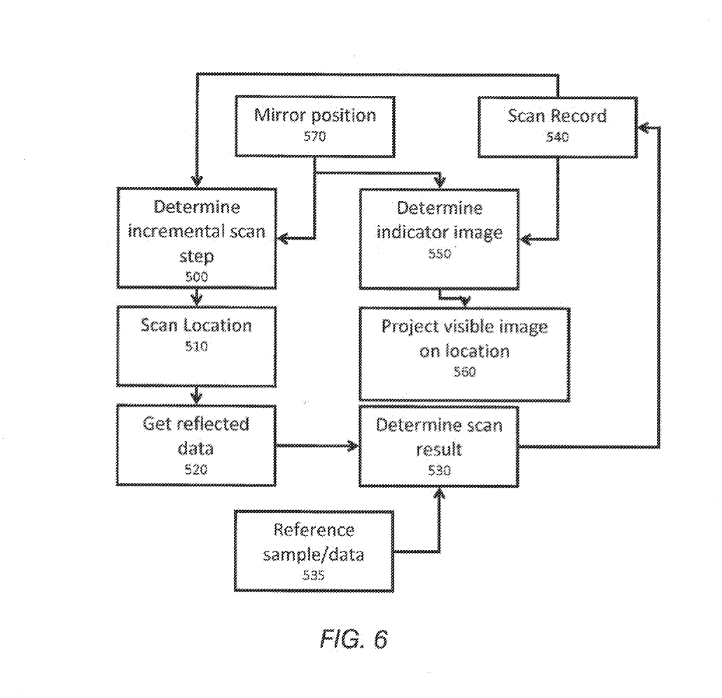

FIG. 6 is a flow chart depicting some embodiments of how the method can be performed and/or employed via a computer. Thus, in some embodiments, the computer will have the coding and/or algorithms for executing one or more of the processes noted in FIG. 5. In some embodiments, one can scan a location, as depicted in block 510. One can then obtain reflected data (block 520) from the location and determine the scan result 530. The scan result 530 can include and/or be compared and/or combined with one or more reference sample results and/or data (block 535). The scan result can optionally be recorded (block 540). This can either result in further processing to determine a subsequent incremental scan step 500, which can then lead to a subsequent scan location (back at block 510), and/or be used to determine an indicator image 550, which can then be used to project a visible light image on the location 560. The mirror position (block 570) can be used for a variety of the processes provided herein, including determining the subsequent incremental scan step (block 500) and determining the indictor image (block 550). The mirror position (block 570) can also be employed in getting and/or determining the scan results (blocks 520 and 530).

In some embodiments, the computing device 470 is configured to synchronize a pulsing of the infrared light source 410 and the visible light source 420 such that only one passes through the probe light guide 440 at a time.

In some embodiments, the computing device 470 controls the visible light source 420. In some embodiments, the computing device 470 electronically pulses the visible light source 420.

The system 400 can be configured such that infrared light generated from the infrared light source 410 passes into the collinear light guide 440, into the probe light guide 440, onto the optical head 450 and onto a sample. The system is further configured such that infrared light external to the optical head 450 can pass onto the optical head 450 and onto the detector 460. Furthermore, the system can be configured such that visible light generated from the visible light source 420 passes into the collinear light guide 440, into the probe light guide 440, onto the optical head 450, to be directed onto the sample in a pattern to indicate the presence of target cells (such as cancerous cells).

A variety of possible IR signatures can be employed in various embodiments herein. For example, as shown in FIGS. 2A-2C, different tissue types can produce distinguishable infrared Raman spectra when irradiated with a beam of infrared light. For example. Raman spectra show four characteristic Raman bands at a Raman shift of about 1078, 1300, 1445 and 1651 cm⁻¹ for an exemplary benign tissue (FIG. 2A), three characteristic Raman bands at a Raman shift of about 1240, 1445, and 1659 cm⁻¹ for an exemplary benign tumor tissue (FIG. 2B), and two characteristic Raman bands at a Raman shift of about 1445 and 1651 cm⁻¹ for an exemplary malignant tumor tissue (FIG. 2C). Thus, this information, and/or other optical information regarding the cells can be used to characterize the cells in regard to different aspects.

In some embodiments, the target cell is part of at least one of a pre-cancerous, benign, or a malignant tumor. In some embodiments, the target cell is a liver cell (that can be cancerous, benign, or malignant). In some embodiments, the target cell is a cell of an internal organ of a subject. In some embodiments, the target cell is a cell on the subject's skin. In some embodiments, the target cell is a cell along the digestive tract of the subject. The present target cells are not to be limited to any particular cell type, unless expressly denoted.

The target cell provides a distinguishable and/or identifiable IR signature. In some embodiments, the target cell is a benign tissue. In some embodiments, the benign tissue (target cell) has four Raman bands. For example, in some embodiments, the target cell has an IR signature including Raman bands at a Raman shift of about 1078, 1300, 1445 and 1651 cm⁻¹. In some embodiments, the target cell is a benign tumor tissue. In some embodiments, the benign tumor tissue (target cell) has three Raman bands. For example, in some embodiments, the target cell has an IR signature including Raman bands at a Raman shift of about 1240, 1445, and 1659 cm⁻¹. In some embodiments, the target cell is a malignant tumor tissue. In some embodiments, the malignant tumor tissue (target cell) has two Raman bands. For example, in some embodiments, the target cell has an IR signature including Raman bands at a Raman shift of about 1445 and 1651 cm⁻¹. In some embodiments, at least a part, if not all, of the full spectrum of the IR signature of the target cell can be used to determine the best match. Thus, rather than looking at localized peaks or wavelengths, partial or full signatures can be used for comparisons and for determining the best match of a given target cell to the various tissue states.

The IR signature of the target cell can be associated with the proteins and/or DNA in and/or on the target cell. In some embodiments, the IR signature of the target cell is different than the IR signature of the one or more cells adjacent to the target cell.

In some embodiments, the signature monitored is from a fluorescent or other molecule that has been added to the subject. Thus, in some embodiments, a detectable marker has been added to the subject, and the probe can be used to detect the detectable marker (which need not be detectable to the human eye), and the system can then detect the detectable marker (and need not employ an IR signature system for the initial detection of the target cell.

As will be appreciated by one of skill in the art, given the present disclosure, the devices and methods disclosed herein can be employed for cancer detection by light generation, collimation, and/or scanning such that the spectroscopy and visible light are automatically overlaid and matched on the target. This can allow for the indication of cancer on a surface with no requirement for 3D modeling or registration and with minimal equipment in the optical head. In some embodiments, the system can allow for in-body liver resections in which malignant cells are indicated for removal in real time, allowing an advantageous surgical margin.

As will also be appreciated by one of skill in the art, given the present disclosure, the visible image generation and IR spectroscopy light can be merged into the same light guide before entering the patient and the scanning for detection and indication of malignancy are both done by the same scanning mirror. This allows a computing device to build a map of cancerous areas and project it onto the work area in a self aligned manner with no modeling or 3D registration as each pixel is simply indicated if that same pixel returns a cancerous signature.

The methods described herein can be employed in real-time and performed in the surgical suite so that the full identification and excising cycle is done one or more times during a single procedure.

In some embodiments, the device is compatible with laparoscopic and endoscopic implementations, allowing for superior tissue resection with maximum tissue reserve.

In some embodiments, the IR scan can be converted into an optical scan. A conventional discriminator using key Raman bands that have been identified for various cancers can be used. For example, it has been reported that specific Raman bands can be used to distinguish various cancerous states; for example, 4-6 bands have particular differential relationships in multiple cancers researched.

The scanning data need not be binary (cancer/no cancer) but can be a probability score. A variety of methods can be used to interpret the wide diversity of malignant cells. For example, a pathologist can provide either IR spectra of each type of cell for a particular patient before the procedure. The pathologist can use the same sensing head to ensure maximal similarity. Thus, the same classifiers can be used. In another example, a device in communication at the end of the endoscope that is doing comparison can have compartments to receive samples of both healthy and malignant cells from the pathologist. The samples of the healthy and malignant cells can be compared with real time spectroscopy of both the patient and the reference cells. This would, for example, allow the reference cells to be matched in temperature, for example, to the patient tissue surface to match fine grain dependences of spectral response to situation. A patient-specific variable temperature, luminosity, etc., scan characterization can be performed by the pathologist and supplied to the surgical team for the scanner to use for classifying cells. The interpretation can be performed in real-time.

In some embodiments, the interpretation is done on a per-pixel basis as is the output. For example, in some embodiments, a patchwork of cancer/no cancer would show up as a patchwork on the patient, allowing the surgeon to use their judgment as to the best way to remove the cancer safely while leaving the most usable tissue.

In some embodiments, the endoscope camera provides a strong light that is strong enough to be clearly visible. For example, a tilting mirror optical head 450 can be used for very high intensity sources and can be used with Red/Green/Blue lasers so that it can also provide needed white light as appropriate. The tilting mirror based scanning projectors do not need focus and can be projected on arbitrary surfaces without loss of sharpness. The distance from device to organ surface or work area can be controlled by the focal length of the spectrometry. In some embodiments, the distance can be less than 4-6 cm. In some embodiments, the distance can be 10-12 cm. In some embodiments, the distance can be from 1 cm to 12 cm.

The spectroscopic dwell time can depend on specifics like tissue reflectance and spectrum detail level. At movie frame rates (24 frames/second) of optical head 450 scanning, each point can be visited 24 times a second and the amount of dwell time can be adjusted by altering the resolution. For example, if a 10×10 grid is used then each pixel is visited for 1% of scanning time split over 24 portions per second. Use of a computing device allows for integration of multiple scans so that e.g. 100 different scans can be assembled into individual spectrographic results equivalent to 100× the dwell length. The resolution and frame rate can be adjusted to accommodate almost any spectroscopy speed as a simple blinking light can be used for an indicator of a “to-excise” area. The system can be configured to offer feedback like a symbol or arrow if it is moved across a surface too quickly to gather data on each pixel location. The tool can be positioned to scan for up to several minutes, and then project a fixed pattern for excision before repeating the process. The tool can also use cameras or other sensors to detect and correct for movement.

The spectroscope can benefit from light level correction due to non-IR light (although this is expected to be minimal due to the wavelength separation)—such correction can easily be done as the visible light levels can be determined for each instance.

In some embodiments, the system includes a picoprojector display, which uses a MEMS mirror to continuously raster scan a display area while three visible input lasers are pulsed on and off in order to write an appropriate image. In some embodiments, a microprojector unit includes light sources and prisms to get the three colors collinear. In some embodiments, the light sources are separated from the scanner, thereby resulting in microprojector unit small enough to fit into an endoscope head, laparoscopic tool, or robot armature.

In some embodiments, rigid tools can be implemented as well for robotic or more common open surgery.

In some embodiments, the system disclosed herein can have both internal (inside surgical site) and external (surgical suite) applications and/or configurations. In some embodiments, the in-body element can be a small tool head with the MEMS scanner. In some embodiments, the endoscopic armature is conventional with a light path and a small number of electrical signals, and the rest of the system sits in the surgical suite. In some embodiments, the surgical suite component includes of a spectrometer, a projector, a dichroic multiplexer capable of putting the visible light and IR spectroscopy signal into the same light guide, and a computing device to handle the projection image by taking input from the spectrometer and using it to create the projected image. In some embodiments, the spectroscope and computing device can be on a wheeled cart and covered for each separate procedure by a sterile plastic cover.

The probe section can employ a few analog voltage inputs for the mirror and light input, thereby allowing for an easily sterilizable head of glass and metal for repeated procedures. The cable or light guide from the spectroscope to cable or optical head can undergo sterilization of a gaseous type between each procedure. The head can be an integral part of the light guide or cable or detachable from it.

### Example 1

### Indicating a Cancerous Cell

The present example outlines how to identify a target cell. A probe having a light guide and a mirror assembly is provided. An IR signature from an area corresponding to one or more cells is received by the mirror assembly and directed to the light guide. The IR signature passes though the light guide to a detector. A visible light image corresponding to the IR signature is generated by a computing device in communication with the detector. The visible light image is directed through the light guide to the mirror assembly, which projects the visible light onto the area corresponding to the one or more cells, thereby indicating the cells with the cancerous IR signature.

The above example can be applied to any of a number of tissues or applications. For example, while the probe based system is especially useful in applications such as liver resection, it can be used in any application where visualization of the relevant aspects is desired. One such example, of how one could employ visible light, is illustrated in FIG. 5, which shows an image projected onto a leg, demonstrating the visibility of the system on a curved surface. The circular shapes would represent the areas of cancerous cells and a perimeter can optionally be added to indicate to the area being scanned. In some embodiments, the image and spectroscopy are automatically aligned by virtue of the collinear alignment before the scanning mirror and the system does not need to have a model or 3D registration.

### Example 2

### System for Guiding and Collecting Light

The present example illustrates an example configuration of a system for guiding and collecting light. An infrared light source is provided. A visible light source is provided. The infrared and visible light sources are connected to an optical controller. A first end of a light guide is placed into optical communication with the optical controller. A second end of the light guide is connected to an optical head. The optical head includes a MEMS scanning mirror assembly. A detector is placed in optical communication with the optical controller and in electrical communication with a computing device. The computing device is connected to the visible light source and configured to serve as a driver of the MEMS scanning mirror assembly.

### Example 3

### Method for Removing a Tumor

A subject is prepared for surgery to remove a tumor in the liver. The system as outlined in Example 2 is provided and the optical head is placed proximally to the surface of the liver. The infrared light source creates IR light which passes through the optical controller, through the light guide, and through the optical head to project the infrared light onto the surface of the subject's liver. The infrared light projected onto the target area produces an infrared signature(s). At least a part of the IR light from the liver is collected by the optical head and directed through the light guide to the detector. The detector provides an electronic depiction of the infrared signature to the computing device which generates a corresponding visible light image (such that target cells (clusters of cancerous cells) indicated from the IR signal are to be indicated as red “ring” images). The red rings are projected, via the optical head onto the surface of the subject's liver, thereby indicating cancerous tissue. A surgeon can then remove the tissue indicated by the red rings, while leaving the tissue where no red rings have been projected, thereby allowing for faster and more efficient removal of cancerous tissue, while still providing a high level of confidence that all of the cancerous tissue has been removed.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

In an illustrative embodiment, any of the operations, processes, etc. described herein can be implemented as computer-readable instructions stored on a computer-readable medium. The computer-readable instructions can be executed by a processor of a mobile unit, a network element, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs). Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

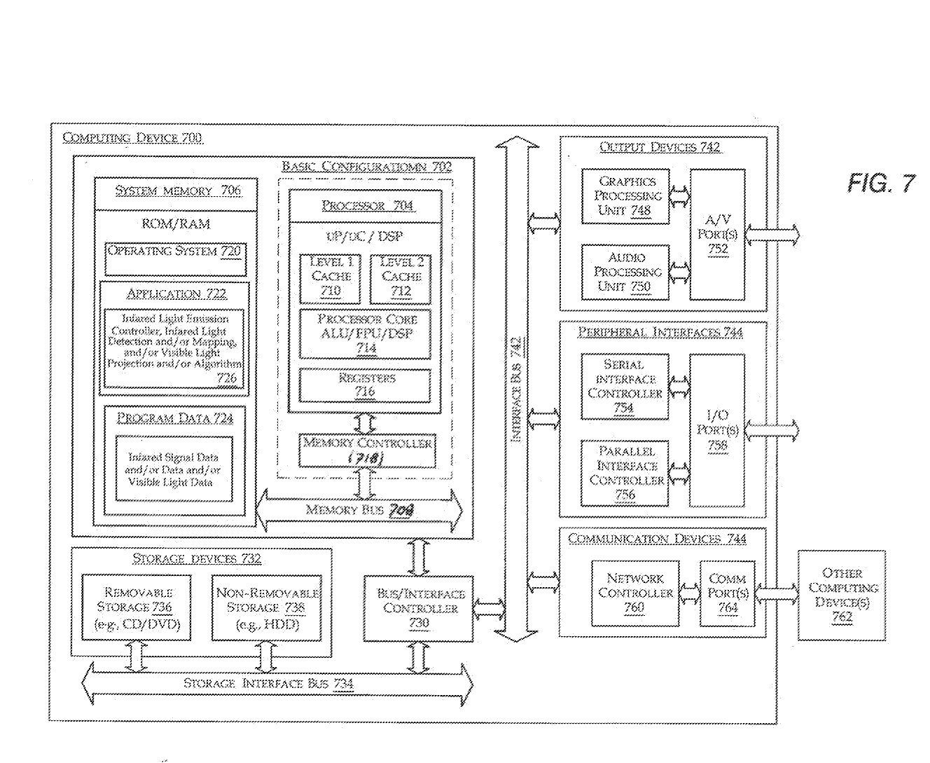

FIG. 7 is a block diagram illustrating an example computing device 700 that is arranged for infrared scanning and indication of target cells in accordance with the present disclosure. In a very basic configuration 702, computing device 700 typically includes one or more processors 704 and a system memory 706. A memory bus 708 may be used for communicating between processor 704 and system memory 706.

Depending on the desired configuration, processor 704 may be of any type including but not limited to a microprocessor (μP), a microcontroller (μC), a digital signal processor (DSP), or any combination thereof. Processor 704 may include one more levels of caching, such as a level one cache 710 and a level two cache 712, a processor core 714, and registers 716. An example processor core 714 may include an arithmetic logic unit (ALU), a floating point unit (FPU), a digital signal processing core (DSP Core), or any combination thereof. An example memory controller 718 may also be used with processor 704, or in some implementations memory controller 718 may be an internal part of processor 704.

Depending on the desired configuration, system memory 706 may be of any type including but not limited to volatile memory (such as RAM), non-volatile memory (such as ROM, flash memory, etc.) or any combination thereof. System memory 706 may include an operating system 720, one or more applications 722, and program data 724. Application 722 may include an infrared light emission controller, infrared light detection and/or mapping, and/or visible light projection method and/or algorithm 726 that is arranged to perform the functions as described herein, including those described with respect to 100, 110, and/or 120 of FIG. 1; 300, 310, 320, 330, 340, 350, 360, 370, 380, 390, and/or 395 of FIG. 3; and/or 500, 510, 520, 570, 550, 560, 530, 535, and/or 540 of FIG. 6. Program data 724 may include infrared signal data and/or visible light data 728 that may be useful for mapping the location of cancerous cells and/or projecting visible light onto the visible cells as is described herein. In some embodiments, application 722 may be arranged to operate with program data 724 on operating system 720 such that infrared light can be projected onto a surface, an infrared signature detected from the surface to determine the location of cancerous areas of the surface and a corresponding map created and projected onto the surface by visible light may be provided as described herein. This described basic configuration 702 is illustrated in FIG. 7 by those components within the inner dashed line.

Computing device 700 may have additional features or functionality, and additional interfaces to facilitate communications between basic configuration 702 and any required devices and interfaces. For example, a bus/interface controller 730 may be used to facilitate communications between basic configuration 702 and one or more data storage devices 732 via a storage interface bus 734. Data storage devices 732 may be removable storage devices 736, non-removable storage devices 738, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 706, removable storage devices 736 and non-removable storage devices 738 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 700. Any such computer storage media may be part of computing device 700.

Computing device 700 may also include an interface bus 740 for facilitating communication from various interface devices (e.g., output devices 742, peripheral interfaces 744, and communication devices 746) to basic configuration 702 via bus/interface controller 730. Example output devices 742 include a graphics processing unit 748 and an audio processing unit 750, which may be configured to communicate to various external devices such as a display or speakers via one or more A/V ports 752. Of course, the light projected onto the subject is also one form of output. Example peripheral interfaces 744 include a serial interface controller 754 or a parallel interface controller 756, which may be configured to communicate with external devices such as input devices (e.g., keyboard, mouse, pen, voice input device, touch input device, IR detector, etc.) or other peripheral devices (e.g., printer, scanner, etc.) via one or more I/O ports 758. An example communication device 746 includes a network controller 760, which may be arranged to facilitate communications with one or more other computing devices 762 over a network communication link via one or more communication ports 764.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 700 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 700 may also be implemented as a personal computer including both laptop computer and non-laptop computer configurations.

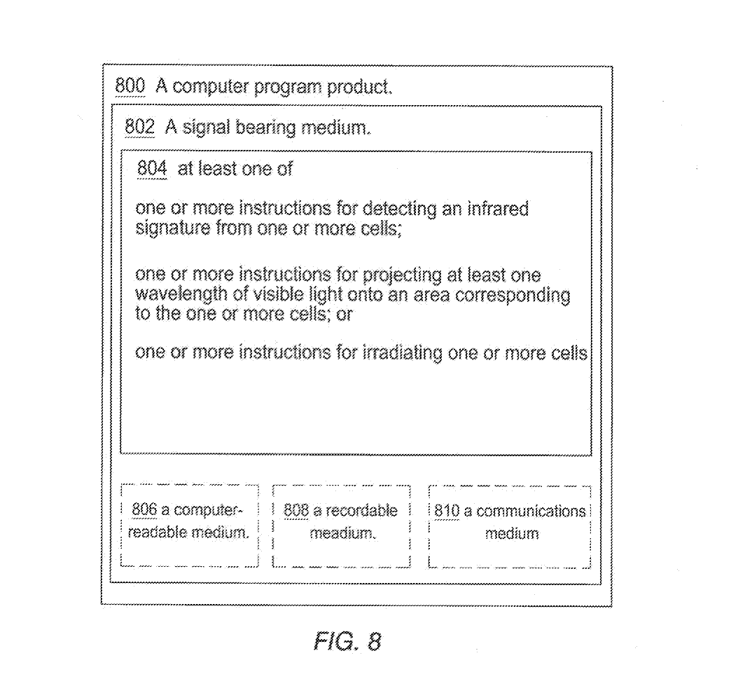

FIG. 8 illustrates an example computer program product 800 arranged in accordance with at least some examples of the present disclosure. Program product 800 may include a signal bearing medium 802. Signal bearing medium 802 may include one or more instructions 804 that, when executed by, for example, a processor, may provide the functionality described above with respect to FIGS. 1, 3, 4, and/or 6. Thus, for example, referring to the system for light manipulation (for example, IR light projection, collection, detection, and/or visible light projection), one or more of modules 500, 510, 520, 535, 530, 560, 550, 540, and 570 may undertake one or more of the blocks shown in FIG. 6 in response to instructions 804 conveyed to the system for light manipulation by medium 802.

In some implementations, signal bearing medium 802 may encompass a computer-readable medium 806, such as, but not limited to, a hard disk drive, a Compact Disc (CD), a Digital Video Disk (DVD), a digital tape, memory, etc. In some implementations, signal bearing medium 802 may encompass a recordable medium 808, such as, but not limited to, memory, read/write (R/W) CDs, R/W DVDs, etc. In some implementations, signal bearing medium 802 may encompass a communications medium 810, such as, but not limited to, a digital and/or an analog communication medium (e.g., a fiber optic cable, a waveguide, a wired communications link, a wireless communication link, etc.). Thus, for example, program product 800 may be conveyed to one or more modules of the system for light manipulation by an RF signal bearing medium 802, where the signal bearing medium 802 is conveyed by a wireless communications medium 810 (e.g., a wireless communications medium conforming with the IEEE 802.11 standard).

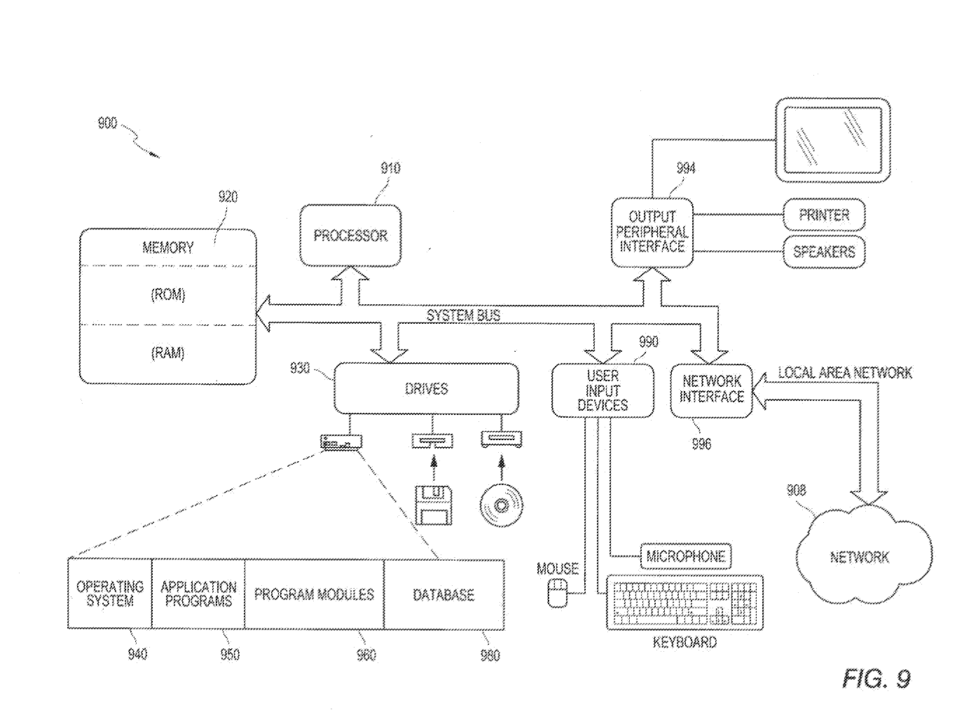

With reference to FIG. 9, depicted is an exemplary computing system for implementing embodiments. FIG. 9 includes a computer 900, including a processor 910, memory 920 and one or more drives 930. The drives 930 and their associated computer storage media, provide storage of computer readable instructions, data structures, program modules and other data for the computer 900. Drives 930 can include an operating system 940, application programs 950, program modules 960, and database 980. Computer 900 further includes user input devices 990 through which a user may enter commands and data. Input devices can include an electronic digitizer, IR detector, mirror system, a microphone, a keyboard and pointing device, commonly referred to as a mouse, trackball or touch pad. Other input devices may include a joystick, game pad, satellite dish, scanner, or the like.

These and other input devices can be connected to processor 910 through a user input interface that is coupled to a system bus, but may be connected by other interface and bus structures, such as a parallel port, game port or a universal serial bus (USB). Computers such as computer 900 may also include other peripheral output devices such as speakers, which may be connected through an output peripheral interface 994 or the like. In some embodiments, the output can also be via the visible light projection components.

Computer 900 may operate in a networked environment using logical connections to one or more computers, such as a remote computer connected to network interface 996 The remote computer may be a personal computer, a server, a router, a network PC, a peer device or other common network node, and can include many or all of the elements described above relative to computer 900. Networking environments are commonplace in offices, enterprise-wide area networks (WAN), local area networks (LAN), intranets and the Internet. For example, in the subject matter of the present application, computer 900 may comprise the source machine from which data is being migrated, and the remote computer may comprise the destination machine or vice versa. Note however, that source and destination machines need not be connected by a network 908 or any other means, but instead, data may be migrated via any media capable of being written by the source platform and read by the destination platform or platforms. When used in a LAN or WLAN networking environment, computer 900 is connected to the LAN through a network interface 996 or an adapter. When used in a WAN networking environment, computer 900 typically includes a modem or other means for establishing communications over the WAN, such as the Internet or network 908. It will be appreciated that other means of establishing a communications link between the computers may be used.

According to one embodiment, computer 900 is connected in a networking environment such that the processor 910 and/or program modules 960 can perform with or as an infrared scanner and projector to indicate cancerous cells in accordance with embodiments herein.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. An endoscopic probe, laparoscopic probe, or endoscopic and laparoscopic probe, the probe comprising:
at least one light guide comprising an input and an output, wherein the at least one light guide allows infrared and visible light to pass through the light guide; and
a mirror assembly in optical communication with the light guide, wherein the mirror assembly is configured to:
(a) direct an infrared beam from the light guide,
(b) receive an infrared signature and direct it into the light guide, and
(c) direct a visible light beam from the light guide.

2. (canceled)

3. (canceled)

4. (canceled)

5. (canceled)

6. (canceled)

7. (canceled)

8. A system for guiding and collecting light, the system comprising:
a probe comprising:
a light guide; and
an optical head detachably connected to the light guide;
a collinear light guide, configured to be in optical communication with the probe;
an infrared (IR) light source, wherein the infrared light source is configured to be in optical, communication with the collinear light guide;
a visible light source, wherein the visible light source is configured to be in detachable, optical, communication with the collinear light guide; and
a detector, wherein the system is configured to allow the detector to detect infrared light that enters the system through the light guide.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A method for indicating a target cell, the method comprising:
detecting an infrared signature from one or more cells; and
projecting at least one wavelength of visible light onto an area corresponding to the one or more cells, thereby indicating a target cell.

29. The method of claim 28, further comprising irradiating the one or more cells with at least one wavelength of infrared light to thereby induce the one or more cells to provide the infrared signature.

30. The method of claim 29, wherein an optical head on a probe is used to irradiate the one or more cells with the at least one wavelength of infrared light.

31. The method of claim 30, wherein the optical head is also used to direct the infrared signature to a detector.

32. The method of claim 31, wherein the optical head is also used to selectively project the at least one wavelength of visible light.

33. The method of claim 32, wherein the optical head comprises a scanning mirror assembly.

34. The method of claim 29, wherein 1) the at least one wavelength of infrared light used for irradiating the one or more cells and 2) the at least one wavelength of visible light, both pass through a same light guide.

35. The method of claim 34, wherein the infrared signature passes through the same light guide.

36. The method of claim 29, wherein the at least one wavelength of infrared light is generated by an infrared light source, and wherein the at least one wavelength of infrared light passes into a collinear light guide, then into a probe light guide, and then to an optical head.

37. The method of claim 36, wherein the infrared signature is directed by the optical head to the probe light guide.

38. The method of claim 37, wherein the infrared signature then passes into a collinear light guide.

39. The method of claim 37, wherein the infrared signature is detected by an infrared detector.

40. The method of claim 37, wherein the at least one wavelength of visible light is generated by a visible light source, and wherein the at least one wavelength of visible light passes into the collinear light guide, then into the probe light guide, and then to the optical head.

41. The method of claim 28, wherein the target cell is part of at least one of a pre-cancerous, benign, or a malignant tumor.

42. The method of claim 28, wherein projecting at least one wavelength of visible light comprises projecting at least a first wavelength of visible light onto a first cell and at least a second wavelength of visible light onto a second cell.

43. The method of claim 42, wherein the first cell is a cancerous cell.

44. The method of claim 43, wherein the second cell is a non-cancerous cell and wherein the second wavelength of light is different from the first wavelength of light.

45. The method of claim 28, wherein a wavelength of the at least one wavelength of visible light corresponds to a size of a cancer cluster.

46. The method of claim 28, wherein a wavelength of the at least one wavelength of visible light corresponds to a depth of a cancerous cell.

47. The method of claim 28, wherein a wavelength of light of the at least one wavelength of visible light is selected so as to contrast with an environment around the one of more cells.

48. The method of claim 28, wherein a wavelength of light of the at least one wavelength of visible light is selected so as to be visibly different from other areas of a subject that are illuminated by other wavelengths of visible light.

49. The method of claim 28, wherein the visible wavelength of light and the infrared wavelength of light are collimated.

50. The method of claim 28, wherein projecting the visible wavelength of light is done via a detachably connected optical head.
